(19) United States
(12) Patent Application Publication
(10) Pub. No.: US 2008/0102194 A1
(43) Pub. Date: May 1, 2008

(54) METHOD AND APPARATUS FOR COATING A MEDICAL DEVICE BY ELECTROLESS PLATING

(75) Inventors: Michael N. Helmus, Worcester, MA (US); Yixin Xu, Newton, MA (US); John Benco, Holliston, MA (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 11/860,866

(22) Filed: Sep. 25, 2007

Related U.S. Application Data
(60) Provisional application No. 60/854,085, filed on Oct. 25, 2006.

(51) Int. Cl. B05D 1/18; A61F 2/06

(52) U.S. Cl. 427/2.25; 623/23.7

(57) ABSTRACT

Methods and apparatus for coating surfaces of medical devices by electroless plating are disclosed. In one embodiment, the invention includes a coating method in which a therapeutic agent and a plating material are plated onto the surface of the medical device by an electroless plating chemical reaction. In another embodiment, a coating method is disclosed in which the coating is formed by suspending a therapeutic agent in a soluble plating solution and plating a plating material onto the medical device by electroless plating wherein the plated material contains the suspended therapeutic agent. In another embodiment, a coating method is provided wherein the coating is formed by initially bonding a therapeutic agent to a plating material, and then plating the bonded therapeutic agent/plating material onto the medical device by electroless plating. In another embodiment, an additive is introduced to the soluble plating solution to regulate the chemical reaction of electroless plating. In another embodiment, a coating method is provided wherein the surface of the medical device is treated, e.g. creating a porous surface layer, to increase the amount of the therapeutic agent that may be plated onto the medical device by electroless plating. The coating is formed by plating a therapeutic agent into and/or onto the porous surface layer. These methods and apparatus are used to apply one or more coating materials, simultaneously or in sequence. In certain embodiments of the invention, the coating materials include therapeutic agents and cationic drugs.

### RELATED APPLICATIONS

This application claims benefit of Provisional Application No. 60/854,085, filed Oct. 25, 2006, which is incorporated herein in its entirety.

### FIELD OF THE INVENTION

The present invention relates to the coating of medical devices.

### BACKGROUND OF THE INVENTION

The positioning and deployment of medical devices within a target site of a patient is a common, often-repeated procedure of contemporary medicine. These devices or implants are used for many medical purposes including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Such medical devices are implanted or otherwise utilized in body lumina and organs such as the coronary vasculature, esophagus, trachea, colon, biliary tract, urinary tract, prostate, brain, and the like.

Coatings are often applied to the surfaces of these medical devices to increase their effectiveness. These coatings may provide a number of benefits including reducing the trauma suffered during the insertion procedure, facilitating the acceptance of the medical device into the target site, and improving the post-procedure effectiveness of the device.

Coating medical devices also provides for the localized delivery of therapeutic agents to target locations within the body, such as to treat localized disease (e.g., heart disease) or occluded body lumens. Such localized drug delivery avoids the problems of systemic drug administration, such as producing unwanted effects on parts of the body which are not to be treated, or not being able to deliver a high enough concentration of therapeutic agent to the afflicted part of the body. Localized drug delivery is achieved, for example, by coating expandable stents, grafts, or balloon catheters, which directly contact the inner vessel wall, with the therapeutic agent to be locally delivered. Stents are often used to support tissue while healing takes place. Expandable stents are tube-like medical devices that often have a mesh-like patterned structure designed to support the inner walls of a lumen. These stents are typically positioned within a lumen and, then, expanded to provide internal support for it. For example, an intraluminal coronary stent may be used during interventional surgery, percutaneous transluminal coronary angioplasty (PTCA), or other heart surgery, to keep the native or grafted vessel open to prevent the reclosure of the blood vessel. The coating on these medical devices may provide for controlled release, which includes long-term or sustained release, of a therapeutic agent.

Aside from facilitating localized drug delivery, medical devices are coated with materials to provide beneficial surface properties. For example, medical devices are often coated with radiopaque materials to allow for fluoroscopic visualization during placement in the body. It is also useful to coat certain devices to achieve enhanced biocompatibility and to improve surface properties such as lubriciousness.

Conventionally, coatings have been applied to medical devices by processes such as dipping and spraying. Dipping and spraying processes usually cannot apply multiple layers of different coatings without requiring appropriate drying time between coating steps, which can increase production time and costs. Further, dipping and spraying processes may result in uneven coating thickness.

There is a need for a cost-effective method for coating the surface of medical devices that results in even and uniform coatings and measured drug doses per unit device.

### SUMMARY OF THE INVENTION

The present invention provides methods and apparatus for coating medical devices by electroless plating of a plating material with a therapeutic agent onto the surface of medical devices. The methods of the present invention permit direct local delivery of therapeutic agents to targeted diseased locations, minimizing waste and loss of expensive therapeutic. The methods also allow the coatings to have uniform thicknesses and mechanical properties, and uniform drug dose.

The present invention regards a method and apparatus for coating at least a portion of a medical device (e.g., a stent). This method includes forming a coating on a bio-compatible medical device by electroless plating of a mixture of a therapeutic agent and a plating material from a soluble plating salt solution onto the surface of the medical device. The electroless plating process may be performed without the use of electrical current.

In another embodiment of the present invention, a method for applying a coating to at least a portion of a bio-compatible medical device is provided wherein the coating is plated onto the surface of the medical device by electroless plating and formed by including additives to the plating solution to regulate the amount of the therapeutic agent and the amount of plating material that is coated.

In another embodiment of the present invention, a method for applying a coating to at least a portion of a bio-compatible medical device is provided wherein the surface of the medical device is treated, e.g. creating a porous surface layer, to increase the amount of the therapeutic agent that may be plated onto the medical device by electroless plating. The coating is formed by plating a therapeutic agent into and/or onto the porous surface layer.

In another embodiment of the present invention, a method for applying a coating to at least a portion of a bio-compatible medical device is provided wherein the coating is formed by suspending a therapeutic agent in a soluble plating solution and plating a material onto the medical device by electroless plating wherein the plating material carries the suspended therapeutic agent such that the coating of plating material contains the suspended therapeutic agent.

In another embodiment of the present invention, a method for applying at least a portion of a coating to a bio-compatible medical device is provided wherein the coating is formed by initially bonding a therapeutic agent to a plating material in a soluble plating solution and then plating the bonded therapeutic agent/plating material onto the medical device by electroless plating such that the coating of plating material contains the bonded therapeutic agent.

In another embodiment of the present invention, an apparatus for applying a coating to a medical device having a surface is provided wherein the coating is formed by plating a mixture of a therapeutic agent and a plating material by electroless plating.

The present invention provides a method and apparatus for coating medical devices having a surface by plating a plating material with a therapeutic agent onto the surface of medical devices by electroless plating. The methods of the present invention permit coating the external surface of the medical devices, which, for example, directly contacts the diseased vessel wall, thereby permitting direct local delivery of therapeutic agents to targeted diseased locations. The methods also minimize wasted coating during the coating process, thereby minimizing the loss of expensive therapeutic. The methods also allow the coatings to have uniform thicknesses and mechanical properties, and uniform drug dose.

An alternate embodiment of the present invention provides for the application of multiple layers of coating material by introducing additives that regulate the amount and/or coating sequence of a first therapeutic agent, a second therapeutic agent, and a plating material to be coated on the medical device. Another alternate embodiment provides for the application of multiple coating layers by altering the acidity or alkalinity of the soluble plating solutions to regulate the amount and/or coating sequence of a plurality of therapeutic agents and plating materials. Another alternate embodiment provides for the application of multiple coating layers by controlling the reaction temperature of the solutions in the electroless plating bath to regulate the amount and/or coating sequence of a plurality of coating materials. These methods of the present invention are time efficient and cost effective because they facilitate the uniform application of multiple layers of coating materials in a single coating process without requiring any intermediate drying step between the application of coating layers. This results in higher process efficiency.

### DETAILED DESCRIPTION

In a first embodiment of the present invention, an apparatus for coating a medical device having a surface is provided. The apparatus in the first embodiment deposits a coating on a medical device by plating a mixture of a reduced therapeutic agent and a plating material by electroless plating. The coating material mixture is plated onto an external surface of a medical device without the need for electrical current. The medical device to be coated can be a stent having a patterned external surface, or any other suitable type of medical device.

The apparatus for coating a medical device by electroless plating includes an electroless plating bath containing at least a solution of a soluble metal plating salt, at least one reducing agent, and a therapeutic agent. In this first embodiment, the therapeutic agent may be ionized for electroless plating of the therapeutic agent with metal from the soluble metal plating salt solution. The therapeutic agent is dissolved into the soluble plating salt solution and dissociated, producing charged drug ions of the therapeutic agent. As one example, the hydrochloride salt of amiloride has an excess of positively charged groups that may be used to be plate onto a substrate medical device. In alternate embodiments, discussed below, the therapeutic agent may be suspended within the mixture of the soluble metal plating salt solution and the reducing agent, and be trapped within the plated metal during the electroless plating process, such that the medical device is coated with the trapped therapeutic agent particles when the metal is plated onto the surface substrate of the medical device.

Referencing the first embodiment, in operation, an electroless plating bath is prepared containing a soluble plating salt solution, at least one chemical reducing agent, and a therapeutic agent. The therapeutic agent may or may not be ionized. It can be a neutral molecule or salt dissolved in solution to provide negative or positive ions or can be reduced by a reducing agent. With the addition of the chemical reducing agent to the soluble plating salt solution and therapeutic agent, an autocatalytic chemical process begins by which the reducing agent reduces the metal and may or may not reduce the therapeutic agent. The soluble metal salt solution may also include a chelating and/or complexing agent to maintain the plating metal in the salt solution.

When a portion of a medical device to be coated is positioned and immersed in the electroless plating bath, a chemical reaction occurs at the surface of the immersed portion of the medical device. Thus, a coating of reduced metal ions and therapeutic ions are electrolessly plated onto the immersed surface of the medical device. As the metal ion concentration decreases during the electroless plating chemical reaction, the rate of deposition may slow. Additional metal salts may be added to the salt solution to replenish the metal ion concentration and maintain a sufficient coating deposition rate. Also, agitation of the medical device and/or salt solution may enhance coating uniformity. In an alternate embodiment, a plurality of reducing agents may be used to individually and separately reduce the metal ions of the soluble plating salt solution and the ions of the therapeutic agent to deposit the metal and therapeutic agent onto the medical device. This can be done sequentially with different baths or simultaneously.

In this first embodiment, the electroless plating process is a chemical reaction in which a reducing agent catalyses the reduction of metal salts to the base metal. Thus, a medical device made from any receptive material surface can be plated by electroless plating, and the material of the medical device need not be electrically conductive to assist in catalyzing the reaction. Medical devices made from non-conductive materials such as ceramics and plastics may be coated by electroless plating. No electrodes, anodes, or voltage sources are needed in electroless plating because the plating is performed without the use of electrical current. Electroless plating allows a relatively constant metal ion concentration to bathe all parts of an immersed medical device. Electroless plating thus enhances coating uniformity because the coating is not dependent on uniformity of the distribution of electrical current, and uniform coating properties may be obtained for any immersed complex shape.

One of ordinary skill in the art will appreciate that the plating rate may be controlled by controlling the plating bath pH levels of alkalinity and acidity, the plating bath temperature, concentrations of the soluble plating salt solution and reducing agent, the presence of any chelating agent, and the amount of agitation of the salt solution and medical device during the electroless plating process. Further, one of ordinary skill in the art will appreciate that the adhesion, porosity, and uniformity of the coating may depend upon the surface preparation of the medical device to be coated. Surface preparation techniques known in the art (e.g. removal of oxide layers) may enhance the receptive nature of the medical device to receive the deposition of the metal and therapeutic agent coating.

One of ordinary skill in the art will also appreciate that the soluble plating salt solution and the chemical reducing agent may be selected from a variety of soluble metal salt solutions and reducing agents. A variety of salt solutions having metal ions of the plating material may be used, depending on the desired metal to be plated onto the medical device, the rate of the chemical reaction, the plating deposition rate, and the desired acidity or alkalinity of the electroless plating bath. As one example, a Pd salt solution of Pd(NH₃)(NO₂)₂, which contains positively charged metal ions, may be used. Solutions containing 5-10 gms/liter of Pd, mixed with a reducing agent, for example a hydrazine or hypophosphite, can produce coatings having a thickness from sub micron to several hundred microns.

In another example, Pd ammine salts (e.g., Pd(NH₃)₄Cl₂) in an alkaline solution may be used to obtain high ductile coatings with low internal stresses at high deposition rates as described in Electroless Plating—Fundamentals and Applications, William Andrew Publishing/Noyes, 1990, ed. Mallory, G. O. and June, B., p. 422. The properties of the deposited coating may be varied from the previously expressed conditions by varying the composition, agitation, temperature, pH, and metal loading. For example, if a high density coating deposition is desired, the metal ion concentration may be raised, and a mild to moderate agitation should be introduced. If a porous or less dense deposition is desired, then these same parameters may be changed in the opposite direction. However, a skilled artisan would appreciate that the acid or salt solution selected should not destroy the dissolved therapeutic agent.

As it is known in the art the general reaction scheme of electroless plating occurs as shown below:

M⁺ⁿ+ne⁻→M⁰+reaction products

### Where the excess electrons are typically provided by a reducing agent.

In yet another example, a soluble metal salt of gold (Au) may be used with a reducing agent such as potassium cynoaurate to produce an autocatalytic chemical reaction in which gold is plated onto a medical device. Handbook of Deposition Technologies for Films and Coatings—Science, Technology and Applications, (2^{nd} ed.), William Andrew Publishing/Noyes, 1994, ed. Bunshah, R. F., p. 600.

One of ordinary skill in the art will appreciate that the reducing agent utilized in the electroless plating process is dependent upon the desired plating metal and therapeutic agent. A variety of chemical reducing agents may be used based on the desired chemical reactions with the soluble metal salt solution and therapeutic agent selected. Some examples of reducing agents, among others, include hydrazines, hypophosphites, amine boranes, borohydrides and formaldehydes. Electroless Plating—Fundamentals and Applications, William Andrew Publishing/Noyes, 1990, ed. Mallory, G. O. and June, B., p. 511.

One of ordinary skill in the art will appreciate that the ionized therapeutic agent utilized in the electroless plating process may be selected from a variety of therapeutic agents. Some examples, among others, of therapeutic agents that may be ionized are cationic drugs, such as amiloride, digoxin, morphine, procainamide, quinidine, quinine, ranitidine, triamterene, trimethoprim, and vancomycin. One of ordinary skill in the art will appreciate that a variety of other acid-stable drugs that may be dissociated into ions may be used. Selection of the drug and plating formulation may be limited to a combination that does not result in the destruction of the drug during the electroless plating process. Further, since electroless plating, when compared to other processes such as sputtering, may be conducted at ambient or relatively low temperatures, the drug will less likely be destroyed.

The medical device may be made from any bio-compatible metal, alloy or synthetic material. Typically, medical devices, e.g. stents, are made from stainless steel, PERSS (a Pt alloyed stainless steel), tantalum, platinum, cobalt chrome alloys, elgiloy or nitinol alloys. However, since the electroless plating process does not require that the device to be plated be an electrically-conductive material, non-conductive bio-compatible materials can also be plated. The plating material can be the same or different metal or alloy as that of the medical device to be coated. Examples of plating materials include, but are not limited to, palladium, platinum, gold, silver, titanium, halfnium, zinc, iridium, aluminum, and niobium, as well as the oxides and alloys of some of those materials.

The medical device, or the portion of the medical device to be coated, is immersed in the electroless plating bath. The medical device may be freely immersed in the bath or secured by a medical device holder. The holder can be, for example, an inflatable balloon or a mandrel which secures the medical device by exerting a force upon the internal surface of the medical device, thereby permitting the external surface to be plated. It will be appreciated by one of ordinary skill in the art that a variety of holder devices can be designed to secure the medical device and permit access to portions of the surface of the medical device.

By holding the medical device from its internal surface with a holder extending the length of the medical device, the holder may mask the internal surface, thereby preventing the coating material from adhering to the internal surface, if desired. Alternatively, if it is desired to coat the entire medical device, the holder may be omitted. Also, a person of ordinary skill in the art will appreciate that the medical device can be masked by a variety of masking methods known in the art to prevent coating certain portions of the medical device. The holder, as one example, can be an inflatable balloon made with any material that is flexible and resilient. Latex, silicone, polyurethane, rubber (including styrene and isobutylene styrene), and nylon, are each examples of materials that may be used in manufacturing the inflatable balloon.

Forming a coating on a medical device by electroless plating a mixture of a therapeutic agent and a plating material may be achieved by several alternative methods. In an alternative embodiment, an additive may be included in the electroless plating bath. The additive may include at least a complexing agent, buffer, bath stabilizer, rate promoter, chelating agent, accelerator, and/or wetting agent.

In another embodiment, the ratio of metal ions in the soluble plating salt solution and the therapeutic agent ions in the therapeutic agent can be varied to control the amount and concentration of the therapeutic agent in the coating. A skilled artisan can appreciate that the ratio of metal to therapeutic agent ions can be controlled, for example, by initially dissolving a greater concentration of therapeutic agent into the electroless plating bath solution.

In another embodiment, a plurality of reducing agents may be used to intermittently plate metal and therapeutic agent coating layers onto the medical device. One of ordinary skill in the art will appreciate that the plating material and therapeutic agent may be selected such that their respective ions are reduced by different reducing agents. Thus, alternating coatings of metal and therapeutic agent layers can be achieved by first adding a first reducing agent for plating metal ions, and then subsequently adding a second reducing agent to plate therapeutic agent ions.

In still another embodiment, two or more therapeutic agents with disparate electroless plating properties may be dissolved and ionized in the electroless plating bath. By adding different reducing agents alternately to individually and separately reduce the plurality of therapeutic agents, multiple coatings of two or more therapeutic agents may be plated.

In yet another embodiment, the surface of the medical device is first treated to create a porous layer to increase the amount of the therapeutic agent that may be electrolessly plated onto the medical device. Thereafter, the coating is formed by electroless plating of the therapeutic agent onto the treated surface and into the pores of the porous layer. Due to the large surface area of the porous structure, large amount of therapeutic agents can be drawn into the pores, and a larger concentration of therapeutic agent can be applied.

The porous layer can be created by several methods, including vapor deposition processes, CVD, PVD, plasma deposition, electroplating, electroless plating, sintering, sputtering or other methods known in the art. The deposited porous material may be the same as the substrate or the metal being plated by electroless plating. The amount of plated drug which can be loaded onto the porous layer is much greater than the amount of plated drug that can be loaded onto a flat surface. This is because the pores not only add more surface area upon which to load the plated drug, but also because the volume of the pores are filled with the plated drug. For example, the surface area of 1 gram of non-porous gold is about 8×10⁻⁵ m²/g, whereas the surface area of nanoporous gold made by a de-alloying process is about 2 m²/g. Although this embodiment described above involves a two-step process, by forming the porous layer first at relatively high temperatures, or annealing the substrate at relatively high temperatures to enhance the adhesion, the second step of therapeutic agent plating can be done at a lower temperature or room temperature. The shape of the pores in the porous surface may serve as a means to control the release rate of the therapeutic agent. For example, a pore with a narrow opening and a wide bottom may release drugs more slowly than a pore with a wide opening and a narrow bottom. Also, a pore with a jagged inner surface, or with varying narrow and wide radiuses throughout the depth of the pore, or a pore with an elongated tortuous passageway may also serve to meter the release rate of the drug.

Alternatively, the process of forming the porous layer and plating the therapeutic agent may be conducted in one step. Since the porous layer can be created by electroless plating, a mixture of the therapeutic agent and porous plating material may be plated onto the medical device by electroless plating in one step similar to the electroless plating process described herein.

Also, the coating density may vary depending on the concentration of the therapeutic agent in the coating layer. If the concentration is relatively high, the coating can be denser. Further, the concentration of the therapeutic agent may be higher at the outer surface of the treated layer than the interior porous layers. Thus, more therapeutic agents may be released first from the outer surface once the device is deployed in a patient, which may be preferred. Thereafter, the release can be slower as the therapeutic agent is released from the interior porous layers. One of ordinary skill in the art will appreciate that the concentration of the therapeutic agent in the coating layer can be varied by increasing or decreasing the porosity of the porous layer, which permits more or less of the therapeutic agent to be plated, upon treating the surface of the medical device.

By first treating the surface of the medical device to create an interconnected porous network layer of coating, therapeutic agents may be released in a slow and controlled manner. The therapeutic agent is released through the path in the metal matrix. Further, by creating a nano-porous layer, the therapeutic agent may be applied without a polymer binder. The treatment process of creating a porous layer is further described in the following pending patent applications: “Functional Coatings and Designs for Medical Implants,” by Weber, Holman, Eidenschink and Chen, application Ser. No. 10/759,605; “Medical Devices Having Nanostructured Regions for Controlled Tissue Biocompatibility and Drug Delivery,” by Helmus, Xu and Ranada, application Ser. No. 11/007,867; and “Method and Apparatus for Coating a Medical Device by Electroplating,” by Helmus and Xu, application Ser. No. 11/007,297. These applications are incorporated by reference herein.

In an alternative embodiment of the present invention, an apparatus for coating a medical device is provided in which the therapeutic agent is not reduced by the reducing agent in an electroless plating chemical reaction. In this alternative embodiment, the therapeutic agent is in the form of particles suspended within the electroless plating bath. Where the desired therapeutic agent or drug coating cannot be dissolved in the plating bath and become ionized, the therapeutic agent or drug may be produced in fine particles, e.g. nano-meter sized particles, and suspended. During the electroless plating process in which the soluble plating salt solution is reduced and a plating material is plated onto the medical device, these particles will become trapped by the plated metal ions, and the suspended drug particles will plate to the medical device-similar to the way that contamination elements are trapped by plating material ions and become plated to a substrate in conventional electroplating processes. The amount of the therapeutic agent or drug particles that are deposited onto the surface of the medical device varies with the concentration of the therapeutic agent or drug suspended in the plating bath. One of ordinary skill in the art will appreciate that particles of two or more therapeutic agents or drugs may be suspended in the plating bath to allow multiple coatings.

In another alternative embodiment of the present invention, an apparatus for coating a medical device is provided in which the therapeutic agent is not reduced by the reducing agent in an electroless plating chemical reaction; however, the therapeutic agent is first attached to the plating material in the soluble plating salt solution. In this alternative embodiment, the therapeutic agent has drug particles that are initially bonded to the metal ions within the soluble plating salt solution before undergoing the plating process in the electroless plating chemical reaction. One of ordinary skill in the art will appreciate that the therapeutic agent may be attached by a variety of chemical bonding methods (e.g. covalent, hydrogen or ionic bonding). During the chemical reaction in the electroless plating process, in which the soluble plating salt solution is reduced, the bonded therapeutic agent/metal plating material is plated onto the medical device.

The medical devices used in conjunction with the present invention include any device amenable to the coating processes described herein. The medical device, or portion of the medical device, to be coated or surface modified may be made of metal, polymers, ceramics, composites or combinations thereof. Whereas the present invention is described herein with specific reference to a vascular stent, other medical devices within the scope of the present invention include any devices which are used, at least in part, to penetrate the body of a patient. Non-limiting examples of medical devices according to the present invention include catheters, guide wires, balloons, filters (e.g., vena cava filters), stents, stent grafts, vascular grafts, intraluminal paving systems, soft tissue and hard tissue implants, such as orthopedic reair plates and rods, joint implants, tooth and jaw implants, metallic alloy ligatures, vascular access ports, artificial heart housings, heart valve struts and stents (used in support of biologic heart valves), aneurysm filling coils and other coiled coil devices, trans myocardial revascularization (“TMR”) devices, percutaneous myocardial revascularization (“PMR”) devices, hypodermic needles, soft tissue clips, holding devices, and other types of medically useful needles and closures, and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like. Any exposed surface of these medical devices may be coated with the methods and apparatus of the present invention.

The coating materials used in conjunction with the present invention are any desired, suitable substances. In some embodiments, the coating materials comprise therapeutic agents, applied to the medical devices alone or in combination with solvents in which the therapeutic agents are at least partially soluble or dispersible or emulsified, and/or in combination with polymeric materials as solutions, dispersions, suspensions, lattices, etc. The solvents may be aqueous or non-aqueous. Coating materials with solvents may be dried or cured, with or without added external heat, after being deposited on the medical device to remove the solvent. The therapeutic agent may be any pharmaceutically acceptable agent such as a non-genetic therapeutic agent, a biomolecule, a small molecule, or cells. The coating on the medical devices may provide for controlled release, which includes long-term or sustained release, of a therapeutic agent.

Exemplary non-genetic therapeutic agents include anti-thrombogenic agents such as heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet aggregation inhibitors such as cilostazol and tick antiplatelet factors; vascular cell growth promoters such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMPs”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNAs encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHYDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

In a preferred embodiment, the polymer is polyacrylic acid available as HYDROPLUS® (Boston Scientific Corporation, Natick, Mass.), and described in U.S. Pat. No. 5,091,205, the disclosure of which is incorporated by reference herein. In a more preferred embodiment, the polymer is a co-polymer of polylactic acid and polycaprolactone.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The release rate of drugs from drug matrix layers is largely controlled, for example, by variations in the polymer structure and formulation, the diffusion coefficient of the matrix, the solvent composition, the ratio of drug to polymer, potential chemical reactions and interactions between drug and polymer, the thickness of the drug adhesion layers and any barrier layers, and the process parameters, e.g., drying, etc. The coating(s) applied by the methods and apparatus of the present invention may allow for a controlled release rate of a coating substance with the controlled release rate including both long-term and/or sustained release.

The coatings of the present invention are applied such that they result in a suitable thickness, depending on the coating material and the purpose for which the coating(s) is applied. It is also within the scope of the present invention to apply multiple layers of polymer coatings onto the medical device. Such multiple layers may contain the same or different therapeutic agents and/or the same or different polymers, which may perform identical or different functions. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

In addition to the previously described coating layers and their purposes, in the present invention the coating layer or layers may be applied for any of the following additional purposes or combination of the following purposes: to alter surface properties such as lubricity, contact angle, hardness, or barrier properties; to improve corrosion, humidity and/or moisture resistance; to improve fatigue, mechanical shock, vibration, and thermal cycling; to change/control composition at surface and/or produce compositionally graded coatings; to apply controlled crystalline coatings; to apply conformal pinhole free coatings; to minimize contamination; to change radiopacity; to impact bio-interactions such as tissue/blood/fluid/cell compatibility, anti-organism interactions (fungus, microbial, parasitic microorganisms), immune response (masking); to control release of incorporated therapeutic agents (agents in the base material, subsequent layers or agents applied using the above techniques or combinations thereof); or any combinations of the above using single or multiple layers.

One of skill in the art will realize that the examples described and illustrated herein are merely illustrative, as numerous other embodiments may be implemented without departing from the spirit and scope of the present invention.

What is claimed is:

1. A method for coating at least a portion of a medical device comprising the steps of:
introducing a soluble plating material solution to a plating bath, wherein the soluble plating material solution comprises a plating material;
dissolving at least one therapeutic agent in the plating bath;
immersing at least a portion of the medical device to be coated into the plating bath; and
plating the dissolved therapeutic agent onto the medical device by electroless plating to form a coating containing the therapeutic agent.

2. The method of claim 1 wherein the step of plating the dissolved therapeutic agent by electroless plating comprises the steps of:
introducing at least one reducing agent to the plating bath; and
mixing the reducing agent with the dissolved therapeutic agent, wherein the reducing agent chemically reacts with the dissolved therapeutic agent.

3. The method of claim 1 further comprising the step of:
plating the plating material onto the medical device by electroless plating to form a coating containing the plating material.

4. The method of claim 3 wherein the step of plating the plating material by electroless plating comprises the steps of:
introducing at least one reducing agent to the plating bath; and
mixing the reducing agent with the soluble plating material solution, wherein the reducing agent chemically reacts with the soluble plating solution.

5. The method of claim 1 further comprising the step of:
introducing at least one additive to the plating bath.

6. The method of claim 1 further comprising the step of treating a surface of the medical device to be coated.

7. The method of claim 6 wherein the step of treating a surface to be coated comprises creating a porous surface layer.

8. The method of claim 7 wherein the porous surface layer is made by vapor deposition, plasma deposition, sintering, sputtering, electroplating, or electroless plating.

9. The method of claim 1 wherein the therapeutic agent is selected from the group consisting of pharmaceutically active compounds, proteins, oligonucleotides, DNA compacting agents, recombinant nucleic acids, gene/vector systems, and nucleic acids.

10. The method of claim 1 wherein the therapeutic agent is a cationic drug.

11. The method of claim 10 wherein the cationic drug is selected from the group consisting of amiloride, digoxin, morphine, procainamide, quinidine, quinine, ranitidine, triamterene, trimethoprim, or vancomycin.

12. The method of claim 1 wherein the medical device is a stent.

13. A bio-compatible medical device for insertion into a body prepared according to the method of claim 1.

14. A method for coating at least a portion of a medical device comprising the steps of:
introducing a soluble plating material solution to a plating bath, wherein the soluble plating material solution comprises a plating material;
suspending a therapeutic agent in the plating bath;
immersing at least a portion of the medical device to be coated into the plating bath; and
plating the plating material onto the medical device by electroless plating to form a coating on the medical device, wherein the coating contains suspended therapeutic agent.

15. The method of claim 14 further comprising the step of treating a surface of the medical device to be coated.

16. The method of claim 15 wherein the step of treating a surface to be coated comprises creating a porous surface layer.

17. The method of claim 14 wherein the therapeutic agent is selected from the group consisting of pharmaceutically active compounds, proteins, oligonucleotides, DNA compacting agents, recombinant nucleic acids, gene/vector systems, and nucleic acids.

18. The method of claim 14 wherein the medical device is a stent.

19. A bio-compatible medical device for insertion into a body prepared according to the method of claim 14.

20. A method for coating at least a portion of a medical device comprising the steps of:
introducing a soluble plating material solution to a plating bath, wherein the soluble plating material solution comprises a plating material;
dissolving a therapeutic agent in the plating bath, wherein the dissolved therapeutic agent bonds to the plating material;
immersing at least a portion of the medical device to be coated into the plating bath; and
plating the plating material onto the medical device by electroless plating to form a coating on the medical device, wherein the coating contains therapeutic agent bonded to the plating material.

21. The method of claim 20 further comprising the step of treating a surface of the medical device to be coated, wherein the step of treating a surface to be coated comprises creating a porous surface layer.

22. A bio-compatible medical device for insertion into a body prepared according to the method of claim 20.
